(19) United States
(12) Patent Application Publication
(10) Pub. No.: US 2014/0364333 A1
(43) Pub. Date: Dec. 11, 2014

(54) Methods for Live Imaging of Cells

(71) Applicant: President and Fellows of Harvard College, Cambridge, MA (US)

(72) Inventors: Chao-ting WU, Brookline, MA (US); Caroline KIM, Cambridge, MA (US)

(21) Appl. No.: 14/317,169

(22) Filed: Jun. 27, 2014

Related U.S. Application Data
(63) Continuation of application No. 14/205,626, filed on Mar. 12, 2014.
(60) Provisional application No. 61/788,315, filed on Mar. 15, 2013.

(51) Int. Cl. C12Q 1/68

(52) U.S. Cl. CPC C12Q 1/6841
USPC 506/9; 435/6.11

(57) ABSTRACT

The present invention relates to methods of hybridizing nucleic acid probes to genomic DNA.

### RELATED APPLICATIONS

This application is a continuation of U.S. application Ser. No. 14/205,626 (pending), filed Mar. 12, 2014, which claims the priority of U.S. Provisional Application No. 61/788,315 filed Mar. 15, 2013 each of which is hereby incorporated herein by reference in its entirety for all purposes.

### STATEMENT OF GOVERNMENT INTERESTS

This invention was made with government support under grant number GM085169 awarded by the NIH and grant number 5DP1GM106412 awarded by the NIH. The government has certain rights in the invention.

### FIELD

The present invention relates in general to the use of oligonucleotide probes to hybridize to genomic DNA, for example, in a chromosome using fluorescence in situ hybridization. The methods described herein are directed to imaging live cells where labeled probes have been attached to target nucleic acids within the cell.

### BACKGROUND

Fluorescence in situ hybridization (FISH) is a powerful technology wherein nucleic acids are targeted by fluorescently labeled probes and then visualized via microscopy. FISH is a single-cell assay, making it especially powerful for the detection of rare events that might be otherwise lost in mixed or asynchronous populations of cells. In addition, because FISH is applied to fixed cell or tissue samples, it can reveal the positioning of chromosomes relative to nuclear, cytoplasmic, and even tissue structures, especially when applied in conjunction with immunofluorescent targeting of cellular components. FISH can also be used to visualize RNA, making it possible for researchers to simultaneously assess gene expression, chromosome position, and protein localization.

Labeled probes in FISH methods bind to a portion of genomic DNA that has separated into two strands. The labeled probe binds to one of the strands. However, methods of hybridizing labeled probes to live cells would be useful in understanding cellular operations.

### SUMMARY

Embodiments of the present disclosure are directed to methods of imaging live cells using labeled probes and in situ hybridization methods. According to standard FISH methods, a portion of double stranded genomic DNA separates and a labeled probe hybridizes to one of the separated strands. The labeled probe can then be imaged. According to one aspect, any number of probes and labels can be used. According to one aspect, any number of probes and labels can be used which are spectrally resolvable. Accordingly, a plurality of probes may be used with different labels. Accordingly, a plurality of probes may be used with different, spectrally resolvable labels. In this manner, one or more or a plurality of genomic nucleic acid sequences may be visualized in a live cell using the methods described herein.

According to one aspect, a method of imaging a live cell by fluorescence in situ hybridization is provided including combining the live cell under growth conditions with a labeled probe having a sequence complementary to a genomic nucleic acid sequence, and imaging the labeled probe within the live cell bound to genomic DNA.

According to one aspect, the probe is a labeled locked nucleic acid probe, a labeled oligonucleotide, an ECHO probe, a molecular beacon, a labeled toe-hold probe, a labeled TALE or a labeled Cas9/RNA complex. According to one aspect, the method further includes the step of removing unhybridized probe from within the live cell. The unhybridized probe may be removed by placing the cell in probe-free growth media and allowing the cell to double once or twice. The unhybridized probe may be removed by centrifugation of the cell and resuspension in probe-free growth media.

According to one aspect, the region of the genome targeted for labeling is undergoing replication, whether naturally occurring replication or replication of the genomic nucleic acid sequence is induced before being contacted by the labeled probe.

According to one aspect, a method of imaging a live cell by fluorescence in situ hybridization is provided including placing the live cell under growth conditions, synthesizing a Cas9 within the cell, synthesizing RNA within the cell to bind genomic DNA and to complex with the Cas9 forming a Cas9/RNA complex, labeling the Cas 9/RNA complex, and imaging the labeled Cas 9/RNA complex within the live cell bound to genomic DNA. According to one aspect, the Cas9 is synthesized in vivo by using an integrated construct, a transiently transfected construct, by injection into the cell of a syncitia of nuclei or via electroporation into cells and/or nuclei.

According to one aspect, the RNA is synthesized in vivo by using an integrated construct, a transiently transfected construct, by injection into the cell of a syncitia of nuclei or via electroporation into cells and/or nuclei. According to one aspect, the Cas9/RNA complex is labeled by making a fusion protein that includes Cas9 and a reporter, by injection of RNA that has been attached to a reporter into the cell or by a syncitia of nuclei including RNA that has been attached to a reporter, by electroporation into cells or nuclei or by indirect labeling of the RNA by hybridization with a labeled secondary oligonucleotide. According to one aspect, the label is a conditional reporter. According to one aspect, the label is a conditional reporter based on the binding of Cas9/RNA to the target nucleic acid. According to one aspect, the label is quenched and is then activated upon the Cas9/RNA complex binding to the target nucleic acid.

According to one aspect, a method of imaging a live cell by fluorescence in situ hybridization is provided including genetically altering the live cell to include one or more nucleic acid sequences complementary to a probe, combining the live cell under growth conditions with the probe having a sequence complementary to the one or more sequences added, and imaging the probe within the live cell bound to one or more sequences added.

According to one aspect, a method of making a live cell for in situ hybridization is provided including genetically altering the live cell to include one or more nucleic acid sequences complementary to a probe.

### BRIEF DESCRIPTION OF THE DRAWINGS

The foregoing and other features and advantages of the present invention will be more fully understood from the following detailed description of illustrative embodiments taken in conjunction with the accompanying drawing in which:

FIG. 1 presents images of live Drosophila cells imaged with a labeled probe.

FIG. 2 presents images of live Drosophila cells imaged with a TAL-GFP and TAF-GFP on fixed cells.

FIG. 3 is a schematic representation of a probe system based on CRISPR.

FIG. 4 is a schematic representation of an alternate probe system based on CRISPR.

FIG. 5 is a schematic representation of an alternate probe system based on CRISPR.

FIG. 6 is a schematic representation of an alternate probe system based on CRISPR.

### DETAILED DESCRIPTION

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

It is to be understood that methods steps described herein need not be performed in the order listed unless expressly stated. Method steps may be performed in any order. Further, method steps may be performed simultaneously or together and need not be performed separately or individually. To the extent that methods describe multiple probes being hybridized to various nucleic acids on separate homologs, such hybridization may be performed as a single step with all reagents combined. Individual hybridization steps need not be performed individually.

According to certain embodiments, a method of imaging a live cell by fluorescence in situ hybridization is provided including combining the live cell under growth conditions with a labeled probe having a sequence complementary to a genomic nucleic acid sequence, and imaging the labeled probe within the live cell bound to DNA or RNA. Accordingly, the methods provide the visualization of nucleic acids, such as DNA or RNA, such as DNA of chromosomes, in live cells. The term “live” cell includes a functioning cell insofar as cellular functions are being carried out. A live cell is distinguished from a dead cell where no cellular functions are being carried out. Those of skill in the art can readily distinguish between a live cell and a dead cell for purposes of the present disclosure.

According to one aspect, exemplary probes are selected that are compatible with a live cell in being non-toxic to the cell. According to one aspect, exemplary probes are selected that are able to withstand degradation that may occur within a live cell due to functioning cellular processes and chemicals, at least to the extent that the probes are capable of hybridizing with genomic DNA, though they may be degraded to a certain extent, i.e. are partially degraded. Probes are also selected that are capable of targeting endogenous DNA or RNA which may be double stranded or associated with proteins or other bound factors. Methods are also selected to reduce the presence of unbound probes within the live cell or otherwise reduce background signals resulting from unbound probes.

According to one aspect, the term “labeled probe” refers to both a single molecule including a probe sequence and a label attached thereto, such as by covalent attachment, or a probe sequence and a separate label component which are added as separate species but then combine to form a labeled probe. Such an embodiment may be referred to as a secondary label. Wherever reference is made to hybridization of a labeled nucleotide, such hybridization may be accomplished with the labeled nucleotide or other labeled compound being part of a hybridization probe.

Exemplary methods useful for imaging live cells include fluorescence in situ hybridization or FISH which is a cytogenetic technique that is used to detect and localize the presence or absence of specific DNA sequences on chromosomes. FISH uses fluorescent probes that bind to only those parts of the chromosome with which they show a high degree of sequence complementarity. Fluorescence microscopy can be used to find out where the fluorescent probe is bound to the chromosomes.

Exemplary FISH methods include standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides. The reagents used in each of these steps and their conditions of use vary depending on the particular situation and whether their use is required with any particular probes.

Hybridization conditions are also described in U.S. Pat. No. 5,447,841. It will be appreciated that numerous variations of in situ hybridization protocols and conditions are known and may be used in conjunction with the present invention by practitioners following the guidance provided herein.

As used herein, the term “chromosome” refers to the support for the genes carrying heredity in a living cell, including DNA, protein, RNA and other associated factors. There exists a conventional international system for identifying and numbering the chromosomes of the human genome. The size of an individual chromosome may vary within a multi-chromosomal genome and from one genome to another. A chromosome can be obtained from any species. A chromosome can be obtained from an adult subject, a juvenile subject, an infant subject, from an unborn subject (e.g., from a fetus, e.g., via prenatal test such as amniocentesis, chorionic villus sampling, and the like or directly from the fetus, e.g., during a fetal surgery) from a biological sample (e.g., a biological tissue, fluid or cells (e.g., sputum, blood, blood cells, tissue or fine needle biopsy samples, urine, cerebrospinal fluid, peritoneal fluid, and pleural fluid, or cells therefrom) or from a cell culture sample (e.g., primary cells, immortalized cells, partially immortalized cells or the like). In certain exemplary embodiments, one or more chromosomes can be obtained from one or more genera including, but not limited to, Homo, Drosophila, Caenorhabiditis, Danio, Cyprinus, Equus, Canis, Ovis, Ocorynchus, Salmo, Bos, Sus, Gallus, Solanum, Triticum, Oryza, Zea, Hordeum, Musa, Avena, Populus, Brassica, Saccharum and the like.

Useful probes within the scope of the present disclosure include labeled locked nucleic acids (LNAs), labeled peptide nucleic acids (PNAs), oligopaints described in US 2010/0304994, toe-hold probes, ECHO probes, molecular beacons, TALE probes, and CRISPR probes.

Locked nucleic acid probes and peptide nucleic acid probes are known to those of skill in the art and are described in Briones et al., Anal Bioanal Chem (2012) 402:3071-3089 hereby incorporated by reference in its entirety.

“Oligopaints” are described in US 2010/0304994 and in Beliveau et al., PNAS (2012). As used herein, the term “Oligopaint” refers to detectably labeled polynucleotides that have sequences complementary to an oligonucleotide sequence, e.g., a portion of a DNA sequence e.g., a particular chromosome or sub-chromosomal region of a particular chromosome. Oligopaints are generated from synthetic probes and arrays that are, optionally, computationally patterned (rather than using natural DNA sequences and/or chromosomes as a template). Since Oligopaints are generated using nucleic acid sequences that are present in a pool, they are no longer spatially addressable (i.e., no longer attached to an array). Surprisingly, however, this method increases resolution of the oligopaints over chromosome paints that are made using yeast artificial chromosomes (YACs), bacterial artificial chromosomes (BACs), and/or flow sorted chromosomes.

In certain exemplary embodiments, small Oligopaints are provided. As used herein, the term “small Oligopaint” refers to an Oligopaint of between about 5 bases and about 100 bases long, or an Oligopaint of about 5 bases, about 10 bases, about 15 bases, about 20 bases, about 25 bases, about 30 bases, about 35 bases, about 40 bases, about 45 bases, about 50 bases, about 55 bases, about 60 bases, about 65 bases, about 70 bases, about 75 bases, about 80 bases, about 85 bases, about 90 bases, about 95 bases, or about 100 bases. Small Oligopaints can access targets that are not accessible to longer oligonucleotide probes. For example, in certain aspects small Oligopaints can pass into a cell, can pass into a nucleus, and/or can hybridize with targets that are partially bound by one or more proteins, etc. Small Oligopaints are also useful for reducing background, as they can be more easily washed away than larger hybridized oligonucleotide sequences. As used herein, the terms “Oligopainted” and “Oligopainted region” refer to a target nucleotide sequence (e.g., a chromosome) or region of a target nucleotide sequence (e.g., a sub-chromosomal region), respectively, that has hybridized thereto one or more Oligopaints. Oligopaints can be used to label a target nucleotide sequence, e.g., chromosomes and sub-chromosomal regions of chromosomes during various phases of the cell cycle including, but not limited to, interphase, preprophase, prophase, prometaphase, metaphase, anaphase, telophase and cytokenesis.

According to certain aspects, labeled toe-hold probes are useful in the methods described herein. Toe-hold probes are known to those of skill in the art as described in Zhang et al., Optimizing the Specificity of Nucleic Acid Hybridization, Nature Chemistry, DOI: 10.1038/NCHEM.1246 (published online Jan. 22, 2012) hereby incorporated by reference in its entirety for all purposes.

Molecular beacons are hairpin shaped molecules with an internally quenched fluorophore whose fluorescence is restored when they bind to a target nucleic acid sequence. Molecular beacons are known to those of skill in the art as described in Guo et al., Anal. Bioanal. Chem. (2012) 402:3115-3125 hereby incorporated by reference in its entirety.

TALEN probes are known to those of skill in the art as described in Joung et al., Nature Reviews/Molecular Cell Biology Vol. 14, pp. 49-55 (2013) hereby incorporated by reference in its entirety.

ECHO probes are sequence-specific, hybridization-sensitive, quencher-free fluorescent probes for RNA detection, which have been designed using the concept of fluorescence quenching caused by intramolecular excitonic interaction of fluorescent dyes. ECHO probes are known to those of skill in the art as described in Kubota et al., PLoS ONE, Vol. 5, Issue 9, e13003 (2010); Okamoto, Chem. Soc. Rev., 2011, 40, 5815-5828, Wang et al., RNA (2012), 18:166-175, each of which are hereby incorporated by reference in their entireties.

CRISPR/Cas systems for nucleic acid binding are known to those of skill in the art and disclosed in Cong et al., Sciencexpress, sciencemag.org, Jan. 3, 2013, 10.1126/science.1231143; Jinek et al., Science, vol. 337, pp. 816-821 (2012) and Mali et al., Sciencexpress, sciencemag.org, Jan. 3, 2013, 10.1126/science.1232033, each of which are hereby incorporated by reference in their entireties.

### Nucleic Acid

The terms “nucleic acid,” “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides or ribonucleotides, or analogs thereof. The labeled probes described herein may include or be a “nucleic acid,” “nucleic acid molecule,” “nucleic acid sequence,” “nucleic acid fragment,” “oligonucleotide” or “polynucleotide.” Oligonucleotides or polynucleotides useful in the methods described herein may comprise natural nucleic acid sequences and variants thereof, artificial nucleic acid sequences, or a combination of such sequences. Oligonucleotides or polynucleotides may be single stranded or double stranded.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

### Nucleotides

The terms “nucleotide analog,” “altered nucleotide” and “modified nucleotide” refer to a non-standard nucleotide, including non-naturally occurring ribonucleotides or deoxyribonucleotides. In certain exemplary embodiments, nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; 0- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

Examples of modified nucleotides include, but are not limited to diaminopurine, S²T, 5-fluorouracil, 5-bromouracil, 5-chlorouracil, 5-iodouracil, hypoxanthine, xantine, 4-acetylcyto sine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Non-naturally occurring nucleotides and polymerases which can be used with such bases in include those described in Gommers-Ampt et al., The FASEB Journal, Vol. 9, pp. 1034-1042 (1995); Leconte, et al., J. Am. Chem. Soc; 127(36), pp. 12470-12471 (2005); Leconte et al., Angew. Chem. Int. Ed. 2010, 49, pp. 5921-5924; Malyshev et al., J. Am. Chem. Soc. 2009, 131, 14620-14621; Metzker, Genome Research 15:1767-1776 (2005); Metzker, Nature Reviews/Genetics, Vol. 11, pp. 31-46 (2010); and Yang et al., Angew. Chem. Int. Ed, 2010, 49, 177-180 each of which is hereby incorporated by reference in its entirety for all purposes.

In certain exemplary embodiments, nucleotide analogs or derivatives will be used, such as nucleosides or nucleotides having protecting groups on either the base portion or sugar portion of the molecule, or having attached or incorporated labels, or isosteric replacements which result in monomers that behave in either a synthetic or physiological environment in a manner similar to the parent monomer. The nucleotides can have a protecting group which is linked to, and masks, a reactive group on the nucleotide. A variety of protecting groups are useful in the invention and can be selected.

### Oligonucleotide Probes

Oligonucleotide sequences, such as single stranded oligonucleotide sequences to be used for labeled probes, may be isolated from natural sources, synthesized or purchased from commercial sources. In certain exemplary embodiments, oligonucleotide sequences may be prepared using one or more of the phosphoramidite linkers and/or sequencing by ligation methods known to those of skill in the art. Oligonucleotide sequences may also be prepared by any suitable method, e.g., standard phosphoramidite methods such as those described herein below as well as those described by Beaucage and Carruthers ((1981) Tetrahedron Lett. 22: 1859) or the triester method according to Matteucci et al. (1981) J. Am. Chem. Soc. 103:3185), or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors.

In certain exemplary embodiments, oligonucleotide sequences may be prepared using a variety of microarray technologies known in the art. Pre-synthesized oligonucleotide and/or polynucleotide sequences may be attached to a support or synthesized in situ using light-directed methods, flow channel and spotting methods, inkjet methods, pin-based methods and bead-based methods set forth in the following references: McGall et al. (1996) Proc. Natl. Acad. Sci. U.S.A. 93:13555; Synthetic DNA Arrays In Genetic Engineering, Vol. 20:111, Plenum Press (1998); Duggan et al. (1999) Nat. Genet. S21:10; Microarrays: Making Them and Using Them In Microarray Bioinformatics, Cambridge University Press, 2003; U.S. Patent Application Publication Nos. 2003/0068633 and 2002/0081582; U.S. Pat. Nos. 6,833,450, 6,830,890, 6,824,866, 6,800,439, 6,375,903 and 5,700,637; and PCT Application Nos. WO 04/031399, WO 04/031351, WO 04/029586, WO 03/100012, WO 03/066212, WO 03/065038, WO 03/064699, WO 03/064027, WO 03/064026, WO 03/046223, WO 03/040410 and WO 02/24597.

Polymerase recognition sites, cleavage sites and/or label or detectable moiety addition sites may be added to the single stranded oligonucleotides during synthesis using known materials and methods.

Nucleic acid probes according to the present disclosure may be labeled or unlabeled. Certain nucleic acid probes may be directly labeled or indirectly labeled.

According to certain aspects, nucleic acid probes may include a primary nucleic acid sequence that is non-hybridizable to a target nucleic acid sequence in addition to the sequence of the probe that hybridizes to the target nucleic acid sequence. Exemplary primary nucleic acid sequences or target non-hybridizing nucleic acid sequences include between about 10 nucleotides to about 100 nucleotides, between about 10 nucleotides to about 70 nucleotides, between about 15 nucleotides to about 50 nucleotides, between about 20 nucleotides to about 60 nucleotides and all ranges and values in between whether overlapping or not. According to certain aspects, the primary nucleic acid sequence is hybridizable with one or more secondary nucleic acid sequences. According to certain aspects, the secondary nucleic acid sequence may include a label. According to this aspect, the nucleic acid probes are indirectly labeled as the secondary nucleic acid binds to the primary nucleic acid thereby indirectly labeling the probe which hybridizes to the target nucleic acid sequence. According to certain aspects, a plurality of nucleic acid probes is provided with each having a common primary nucleic acid sequence. That is, the primary nucleic acid sequence is common to a plurality of nucleic acid probes, such that each nucleic acid probe in the plurality has the same or substantially similar primary nucleic acid sequence. According to one aspect, the primary nucleic acid sequence is a single sequence species. In this manner, a plurality of common secondary nucleic acid sequences is provided which hybridize to the plurality of common primary nucleic acid sequences. That is, each secondary nucleic acid sequence has the same or substantially similar nucleic acid sequence. According to one exemplary embodiment, a single primary nucleic acid sequence is provided for each of the nucleic acid probes in the plurality. Accordingly, only a single secondary nucleic acid sequence which is hybridizable to the primary nucleic acid sequence need be provided to label each of the nucleic acid probes. According to certain aspects, the common secondary nucleic acid sequences may include a common label. According to this aspect, a plurality of nucleic acid probes are provided having substantially diverse nucleic acid sequences hybridizable to different target nucleic acid sequences and where the plurality of nucleic acid probes have common primary nucleic acid sequences. Accordingly, a common secondary nucleic acid sequence having a label may be used to indirectly label each of the plurality of nucleic acid probes. According to this aspect, a single or common primary nucleic acid sequence and secondary nucleic acid sequence pair can be used to indirectly label diverse nucleic acid probe sequences. Such an embodiment is provided where a plurality of nucleic acid probes having primary nucleic acid sequences are commercially synthesized, such as on an array. Labeled secondary nucleic acid sequences can also be commercially synthesized so that they are hybridizable with the primary nucleic acid sequences. The nucleic acid probes may be combined with the labeled secondary nucleic acids and one or more or a plurality of target nucleic acid sequences under conditions such that the nucleic acid probe or probes hybridize to the target nucleic acid sequence or sequences while the primary nucleic acid sequence is nonhybridizable to the target nucleic acid sequence or sequences. A labeled secondary nucleic acid sequence hybridizes with a corresponding primary nucleic acid sequence to indirectly label the nucleic acid probe, thereby labeling the target nucleic acid sequence. According to one aspect, the nucleic acid probes may be combined with the labeled secondary nucleic acids and one or more or a plurality of target nucleic acid sequences together in a one pot method. According to one aspect, the nucleic acid probes may be combined with the labeled secondary nucleic acids and one or more or a plurality of target nucleic acid sequences sequentially, such as the nucleic acid probes are combined with the target nucleic acid to form a mixture and then the labeled secondary nucleic acid is combined with the mixture or the nucleic acid probes are combined with the labeled secondary nucleic acids to form a mixture and then the target nucleic acid is combined with the mixture.

According to certain aspects, the primary nucleic acid sequence is modifiable with one or more labels. According to this aspect, one or more labels may be added to the primary nucleic acid sequence using methods known to those of skill in the art.

According to an additional embodiment, nucleic acid probes may include a first half of a ligand-ligand binding pair, such as biotin-avidin. Such nucleic acid probes may or may not include a primary nucleic acid sequence. The first half of a ligand-ligand binding pair may be attached directly to the nucleic acid probe. According to certain aspects, a second half of the ligand-ligand binding pair may include a label. Accordingly, the nucleic acid probe may be indirectly labeled by the use of a ligand-ligand binding pair. According to certain aspects, a common ligand-ligand binding pair may be used with a plurality of nucleic acid probes of different nucleic acid sequences. Accordingly, a single species of ligand-ligand binding pair may be used to indirectly label a plurality of different nucleic acid probe sequences. The common ligand-ligand binding pair may include a common label or a plurality of common ligand-ligand binding pairs may be labeled with different labels. Accordingly, a plurality of nucleic acid probes of different nucleic acid sequences may be labeled with a single species of label using a single species of a ligand-ligand binding pair.

According to one aspect, the primary nucleic acid sequences may include one or more subsequences that are hybridizable with one or more different secondary nucleic sequences. The one or more secondary nucleic acid sequences may include one or more subsequences that hybridize with one or more tertiary nucleic acid sequences, and so on. Each of the primary nucleic acid sequences, the secondary nucleic acid sequences, the tertiary nucleic acid sequences and so on may be directly labeled with a label or may be indirectly labeled with a label. In this manner, an exponential labeling of the nucleic acid probe can be achieved.

### Labels

A label according to the present disclosure includes a functional moiety directly or indirectly attached or conjugated to a nucleic acid which provides a desired function. According to certain aspects, a label may be used for detection. Detectable labels or moieties are known to those of skill in the art. According to certain aspects, a label may be used to retrieve a particular molecule. Retrievable labels or moieties are known to those of skill in the art. According to certain aspects, a label may be used to target a particular molecule to a target nucleic acid of interest for a desired function. Targeting labels or moieties are known to those of skill in the art. According to certain aspects, a label may be used to react with a target nucleic acid of interest. Reactive labels or moieties are known to those of skill in the art. According to certain aspects, a label may be an antibody, ligand, hapten, radioisotope, therapeutic agent and the like.

As used herein, the term “retrievable moiety” refers to a moiety that is present in or attached to a polynucleotide that can be used to retrieve a desired molecule or factors bound to a desired molecule (e.g., one or more factors bound to a targeting moiety). As used herein, the term “retrievable label” refers to a label that is attached to a polynucleotide (e.g., an Oligopaint) and can, optionally, be used to specifically and/or nonspecifically bind a target protein, peptide, DNA sequence, RNA sequence, carbohydrate or the like at or near the nucleotide sequence to which one or more Oligopaints have hybridized. In certain aspects, target proteins include, but are not limited to, proteins that are involved with gene regulation such as, e.g., proteins associated with chromatin (See, e.g., Dejardin and Kingston (2009) Cell 136:175), proteins that regulate (upregulate or downregulate) methylation, proteins that regulate (upregulate or downregulate) histone acetylation, proteins that regulate (upregulate or downregulate) transcription, proteins that regulate (upregulate or downregulate) post-transcriptional regulation, proteins that regulate (upregulate or downregulate) RNA transport, proteins that regulate (upregulate or downregulate) mRNA degradation, proteins that regulate (upregulate or downregulate) translation, proteins that regulate (upregulate or downregulate) post-translational modifications and the like.

As used herein, the term “targeting moiety” refers to a moiety that is present in or attached to a polynucleotide that can be used to specifically and/or nonspecifically bind one or more factors that associate with, modify or otherwise interact with a nucleic acid sequence of interest (e.g., DNA (e.g., nuclear, mitochondrial, transfected and the like) and/or RNA), including, but not limited to, a protein, a peptide, a DNA sequence, an RNA sequence, a carbohydrate, a lipid, a chemical moiety or the like at or near the nucleotide sequence of interest to which the polynucleotide has hybridized. In certain aspects, factors that associate with a nucleic acid sequence of interest include, but are not limited to histone proteins (e.g., H1, H2A, H2B, H3, H4 and the like, including monomers and oligomers (e.g., dimers, tetramers, octamers and the like)) scaffold proteins, transcription factors, DNA binding proteins, DNA repair factors, DNA modification proteins (e.g., acetylases, methylases and the like).

In other aspects, factors that associate with, modify or otherwise interact with a nucleic acid sequence of interest are proteins including, but not limited to, proteins that are involved with gene regulation such as, e.g., proteins associated with chromatin (See, e.g., Dejardin and Kingston (2009) Cell 136:175), proteins that regulate (upregulate or downregulate) methylation, proteins that regulate (upregulate or downregulate) acetylation, proteins that regulate (upregulate or downregulate) histone acetylation, proteins that regulate (upregulate or downregulate) transcription, proteins that regulate (upregulate or downregulate) post-transcriptional regulation, proteins that regulate (upregulate or downregulate) RNA transport, proteins that regulate (upregulate or downregulate) mRNA degradation, proteins that regulate (upregulate or downregulate) translation, proteins that regulate (upregulate or downregulate) post-translational modifications and the like.

In certain aspects, a targeting and/or retrievable moiety is activatable. As used herein, the term “activatable” refers to a targeting and/or retrievable moiety that is inert (i.e., does not bind a target) until activated (e.g., by exposure of the activatable, targeting and/or retrievable moiety to light, heat, one or more chemical compounds or the like). In other aspects, a targeting and/or retrievable moiety can bind one or more targets without the need for activation of the targeting and/or retrievable moiety. Exemplary methods for attaching proteins, lipids, carbohydrates, nucleic acids and the like are known to those of skill in the art. In certain aspects, a targeting moiety can be a non-targeting moiety that is cross-linked or otherwise modified to bind one or more factors that associate with, modify or otherwise interact with a nucleic acid sequence.

In certain exemplary embodiments, a targeting moiety, a retrievable moiety and/or polynucleotide has a detectable label bound thereto. As used herein, the term “detectable label” refers to a label that can be used to identify a target (e.g., a factor associated with a nucleic acid sequence of interest, a chromosome or a sub-chromosomal region). Typically, a detectable label is attached to the 3′- or 5′-end of a polynucleotide. Alternatively, a detectable label is attached to an internal portion of an oligonucleotide. Detectable labels may vary widely in size and compositions; the following references provide guidance for selecting oligonucleotide tags appropriate for particular embodiments: Brenner, U.S. Pat. No. 5,635,400; Brenner et al., Proc. Natl. Acad. Sci., 97: 1665; Shoemaker et al. (1996) Nature Genetics, 14:450; Morris et al., EP Patent Pub. 0799897A1; Wallace, U.S. Pat. No. 5,981,179; and the like.

Methods for incorporating detectable labels into nucleic acid probes are well known. Typically, detectable labels (e.g., as hapten- or fluorochrome-conjugated deoxyribonucleotides) are incorporated into a nucleic acid, such as a nucleic acid probe during a polymerization or amplification step, e.g., by PCR, nick translation, random primer labeling, terminal transferase tailing (e.g., one or more labels can be added after cleavage of the primer sequence), and others (see Ausubel et al., 1997, Current Protocols In Molecular Biology, Greene Publishing and Wiley-Interscience, New York).

In certain aspects, a suitable targeting moiety, retrievable moiety or detectable label includes, but is not limited to, a capture moiety such as a hydrophobic compound, an oligonucleotide, an antibody or fragment of an antibody, a protein, a peptide, a chemical cross-linker, an intercalator, a molecular cage (e.g., within a cage or other structure, e.g., protein cages, fullerene cages, zeolite cages, photon cages, and the like), or one or more elements of a capture pair, e.g., biotin-avidin, biotin-streptavidin, NHS-ester and the like, a thioether linkage, static charge interactions, van der Waals forces and the like (See, e.g., Holtke et al., U.S. Pat. Nos. 5,344,757; 5,702,888; and U.S. Pat. No. 5,354,657; Huber et al., U.S. Pat. No. 5,198,537; Miyoshi, U.S. Pat. No. 4,849,336; Misiura and Gait, PCT publication WO 91/17160). In certain aspects, a suitable targeting label, retrievable label or detectable label is an enzyme (e.g., a methylase and/or a cleaving enzyme). In one aspect, an antibody specific against the enzyme can be used to retrieve or detect the enzyme and accordingly, retrieve or detect an oligonucleotide sequence or factor attached to the enzyme. In another aspect, an antibody specific against the enzyme can be used to retrieve or detect the enzyme and, after stringent washes, retrieve or detect a factor or first oligonucleotide sequence that is hybridized to a second oligonucleotide sequence having the enzyme attached thereto.

Biotin, or a derivative thereof, may be used as an oligonucleotide label (e.g., as a targeting moiety, retrievable moiety and/or a detectable label), and subsequently bound by a avidin/streptavidin derivative (e.g., detectably labelled, e.g., phycoerythrin-conjugated streptavidin), or an anti-biotin antibody (e.g., a detectably labelled antibody). Digoxigenin may be incorporated as a label and subsequently bound by a detectably labelled anti-digoxigenin antibody (e.g., a detectably labelled antibody, e.g., fluoresceinated anti-digoxigenin). An aminoallyl-dUTP residue may be incorporated into an oligonucleotide and subsequently coupled to an N-hydroxy succinimide (NHS) derivatized fluorescent dye. In general, any member of a conjugate pair may be incorporated into a retrievable moiety and/or a detectable label provided that a detectably labelled conjugate partner can be bound to permit detection. As used herein, the term antibody refers to an antibody molecule of any class, or any sub-fragment thereof, such as an Fab.

Other suitable labels (targeting moieties, retrievable moieties and/or detectable labels) include, but are not limited to, fluorescein (FAM), digoxigenin, dinitrophenol (DNP), dansyl, biotin, bromodeoxyuridine (BrdU), hexahistidine (6xHis), phosphor-amino acids (e.g. P-tyr, P-ser, P-thr) and the like. In one embodiment the following hapten/antibody pairs are used for reaction, retrieval and/or detection: biotin/a-biotin, digoxigenin/a-digoxigenin, dinitrophenol (DNP)/α-DNP, 5-Carboxyfluorescein (FAM)/α-FAM.

Additional suitable labels (targeting moieties, retrievable moieties and/or detectable labels) include, but are not limited to, chemical cross-linking agents. Cross-linking agents typically contain at least two reactive groups that are reactive towards numerous groups, including, but not limited to, sulfhydryls and amines, and create chemical covalent bonds between two or more molecules. Functional groups that can be targeted with cross-linking agents include, but are not limited to, primary amines, carboxyls, sulfhydryls, carbohydrates and carboxylic acids. Protein molecules have many of these functional groups and therefore proteins and peptides can be readily conjugated using cross-linking agents. Cross-linking agents are well known in the art and are commercially available (Thermo Scientific (Rockford, Ill.)).

A detectable moiety, label or reporter can be used to detect a nucleic acid or nucleic acid probe as described herein. Oligonucleotide probes or nucleic acid probes described herein can be labeled in a variety of ways, including the direct or indirect attachment of a detectable moiety such as a fluorescent moiety, hapten, colorimetric moiety and the like. A location where a label may be attached is referred to herein as a label addition site or detectable moiety addition site and may include a nucleotide to which the label is capable of being attached. One of skill in the art can consult references directed to labeling DNA. Examples of detectable moieties include various radioactive moieties, enzymes, prosthetic groups, fluorescent markers, luminescent markers, bioluminescent markers, metal particles, protein-protein binding pairs, protein-antibody binding pairs and the like. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, phycoerythrin and the like. Examples of bioluminescent markers include, but are not limited to, luciferase (e.g., bacterial, firefly, click beetle and the like), luciferin, aequorin and the like. Examples of enzyme systems having visually detectable signals include, but are not limited to, galactosidases, glucorinidases, phosphatases, peroxidases, cholinesterases and the like. Identifiable markers also include radioactive compounds such as ¹²⁵I, ³⁵S, ¹⁴C or ³H. Identifiable markers are commercially available from a variety of sources.

Fluorescent labels and their attachment to nucleotides and/or oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Particular methodologies applicable to the invention are disclosed in the following sample of references: U.S. Pat. Nos. 4,757,141, 5,151,507 and 5,091,519. In one aspect, one or more fluorescent dyes are used as labels for labeled target sequences, e.g., as disclosed by U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); Lee et al.; U.S. Pat. No. 5,066,580 (xanthine dyes); U.S. Pat. No. 5,688,648 (energy transfer dyes); and the like. Labeling can also be carried out with quantum dots, as disclosed in the following patents and patent publications: U.S. Pat. Nos. 6,322,901, 6,576,291, 6,423,551, 6,251,303, 6,319,426, 6,426,513, 6,444,143, 5,990,479, 6,207,392, 2002/0045045 and 2003/0017264. As used herein, the term “fluorescent label” includes a signaling moiety that conveys information through the fluorescent absorption and/or emission properties of one or more molecules. Such fluorescent properties include fluorescence intensity, fluorescence lifetime, emission spectrum characteristics, energy transfer, and the like.

Commercially available fluorescent nucleotide analogues readily incorporated into nucleotide and/or oligonucleotide sequences include, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.) and the like. Alternatively, the above fluorophores and those mentioned herein may be added during oligonucleotide synthesis using for example phosphoroamidite or NHS chemistry. Protocols are known in the art for custom synthesis of nucleotides having other fluorophores (See, Henegariu et al. (2000) Nature Biotechnol. 18:345). 2-Aminopurine is a fluorescent base that can be incorporated directly in the oligonucleotide sequence during its synthesis. Nucleic acid could also be stained, a priori, with an intercalating dye such as DAPI, YOYO-1, ethidium bromide, cyanine dyes (e.g. SYBR Green) and the like.

Other fluorophores available for post-synthetic attachment include, but are not limited to, ALEXA FLUOR™ 350, ALEXA FLUOR™ 405, ALEXA FLUOR™ 430, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, Pacific Orange, rhodamine 6G, rhodamine green, rhodamine red, tetramethyl rhodamine, Texas Red (available from Molecular Probes, Inc., Eugene, Oreg.), Cy2, Cy3, Cy3.5, Cy5, Cy5.5, Cy7 (Amersham Biosciences, Piscataway, N.J.) and the like. FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), APC-Alexa dyes and the like.

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver or gold particles may be used to enhance signal from fluorescently labeled nucleotide and/or oligonucleotide sequences (Lakowicz et al. (2003) BioTechniques 34:62).

Biotin, or a derivative thereof, may also be used as a label on a nucleotide and/or an oligonucleotide sequence, and subsequently bound by a detectably labeled avidin/streptavidin derivative (e.g. phycoerythrin-conjugated streptavidin), or a detectably labeled anti-biotin antibody. Biotin/avidin is an example of a ligand-ligand binding pair. An antibody/antigen binging pair may also be used with methods described herein. Other ligand-ligand binding pairs or conjugate binding pairs are well known to those of skill in the art. Digoxigenin may be incorporated as a label and subsequently bound by a detectably labeled anti-digoxigenin antibody (e.g. fluoresceinated anti-digoxigenin). An aminoallyl-dUTP or aminohexylacrylamide-dCTP residue may be incorporated into an oligonucleotide sequence and subsequently coupled to an N-hydroxy succinimide (NHS) derivatized fluorescent dye. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for an oligonucleotide sequence may include fluorescein (FAM, FITC), digoxigenin, dinitrophenol (DNP), dansyl, biotin, bromodeoxyuridine (BrdU), hexahistidine (6xHis), phosphor-amino acids (e.g. P-tyr, P-ser, P-thr) and the like. In one embodiment the following hapten/antibody pairs are used for detection, in which each of the antibodies is derivatized with a detectable label: biotin/a-biotin, digoxigenin/a-digoxigenin, dinitrophenol (DNP)/α-DNP, 5-Carboxyfluorescein (FAM)/α-FAM.

In certain exemplary embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, PCT publication WO 91/17160 and the like. Many different hapten-capture agent pairs are available for use. Exemplary haptens include, but are not limited to, biotin, des-biotin and other derivatives, dinitrophenol, dansyl, fluorescein, CY5, digoxigenin and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

According to certain aspects, detectable moieties described herein are spectrally resolvable. “Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry: Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

In certain embodiments, the detectable moieties can provide higher detectability when used with an electron microscope, compared with common nucleic acids. Moieties with higher detectability are often in the group of metals and organometals, such as mercuric acetate, platinum dimethylsulfoxide, several metal-bipyridyl complexes (e.g. osmium-bipy, ruthenium-bipy, platinum-bipy). While some of these moieties can readily stain nucleic acids specifically, linkers can also be used to attach these moieties to a nucleic acid. Such linkers added to nucleotides during synthesis are acrydite- and a thiol-modified entities, amine reactive groups, and azide and alkyne groups for performing click chemistry. Some nucleic acid analogs are also more detectable such as gamma-adenosine-thiotriphosphate, iododeoxycytidine-triphosphate, and metallonucleosides in general (see Dale et al., Proc. Nat. Acad. Sci. USA, Vol. 70, No. 8, pp. 2238-2242 (1973)). The modified nucleotides are added during synthesis. Synthesis may refer by example to solid support synthesis of oligonucleotides. In this case, modified nucleic acids, which can be a nucleic acid analog, or a nucleic acid modified with a detectable moiety, or with an attachment chemistry linker, are added one after each other to the nucleic acid fragments being formed on the solid support, with synthesis by phosphoramidite being the most popular method. Synthesis may also refer to the process performed by a polymerase while it synthesizes the complementary strands of a nucleic acid template. Certain DNA polymerases are capable of using and incorporating nucleic acids analogs, or modified nucleic acids, either modified with a detectable moiety or an attachment chemistry linker to the complementary nucleic acid template.

Detection method(s) used will depend on the particular detectable labels used in the reactive labels, retrievable labels and/or detectable labels. In certain exemplary embodiments, target nucleic acids such as chromosomes and sub-chromosomal regions of chromosomes during various phases of the cell cycle including, but not limited to, interphase, preprophase, prophase, prometaphase, metaphase, anaphase, telophase and cytokinesis, having one or more reactive labels, retrievable labels, or detectable labels bound thereto by way of the probes described herein may be selected for and/or screened for using a microscope, a spectrophotometer, a tube luminometer or plate luminometer, x-ray film, a scintillator, a fluorescence activated cell sorting (FACS) apparatus, a microfluidics apparatus or the like.

When fluorescently labeled targeting moieties, retrievable moieties, or detectable labels are used, fluorescence photomicroscopy can be used to detect and record the results of in situ hybridization using routine methods known in the art. Alternatively, digital (computer implemented) fluorescence microscopy with image-processing capability may be used. Two well-known systems for imaging FISH of chromosomes having multiple colored labels bound thereto include multiplex-FISH (M-FISH) and spectral karyotyping (SKY). See Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes) for a review of methods for painting chromosomes and detecting painted chromosomes.

In certain exemplary embodiments, images of fluorescently labeled chromosomes are detected and recorded using a computerized imaging system such as the Applied Imaging Corporation CytoVision System (Applied Imaging Corporation, Santa Clara, Calif.) with modifications (e.g., software, Chroma 84000 filter set, and an enhanced filter wheel). Other suitable systems include a computerized imaging system using a cooled CCD camera (Photometrics, NU200 series equipped with Kodak KAF 1400 CCD) coupled to a Zeiss Axiophot microscope, with images processed as described by Ried et al. (1992) Proc. Natl. Acad. Sci. USA 89:1388). Other suitable imaging and analysis systems are described by Schrock et al., supra; and Speicher et al., supra.

In situ hybridization methods using probes described herein can be performed on a variety of biological or clinical samples, in cells that are in any (or all) stage(s) of the cell cycle (e.g., mitosis, meiosis, interphase, G0, G1, S and/or G2). Examples include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. Samples are prepared for assays of the invention using conventional techniques, which typically depend on the source from which a sample or specimen is taken. These examples are not to be construed as limiting the sample types applicable to the methods and/or compositions described herein.

In certain exemplary embodiments, probes include multiple chromosome-specific probes, which are differentially labeled (i.e., at least two of the chromosome-specific probes are differently labeled). Various approaches to multi-color chromosome painting have been described in the art and can be adapted to the present invention following the guidance provided herein. Examples of such differential labeling (“multicolor FISH”) include those described by Schrock et al. (1996) Science 273:494, and Speicher et al. (1996) Nature Genet. 12:368). Schrock et al. describes a spectral imaging method, in which epifluorescence filter sets and computer software is used to detect and discriminate between multiple differently labeled DNA probes hybridized simultaneously to a target chromosome set. Speicher et al. describes using different combinations of 5 fluorochromes to label each of the human chromosomes (or chromosome arms) in a 27-color FISH termed “combinatorial multifluor FISH”). Other suitable methods may also be used (see, e.g., Ried et al., 1992, Proc. Natl. Acad. Sci. USA 89:1388-92).

Hybridization of the labeled probes described herein to target chromosomes sequences can be accomplished by standard in situ hybridization (ISH) techniques (see, e.g., Gall and Pardue (1981) Meth. Enzymol. 21:470; Henderson (1982) Int. Review of Cytology 76:1). Generally, ISH comprises the following major steps: (1) fixation of the biological structure to be analyzed (e.g., a chromosome spread), (2) pre-hybridization treatment of the biological structure to increase accessibility of target DNA (e.g., denaturation with heat or alkali), (3) optional pre-hybridization treatment to reduce nonspecific binding (e.g., by blocking the hybridization capacity of repetitive sequences), (4) hybridization of the mixture of nucleic acids to the nucleic acid in the biological structure or tissue; (5) post-hybridization washes to remove nucleic acid fragments not bound in the hybridization and (6) detection of the hybridized labelled oligonucleotides (e.g., hybridized Oligopaints). The reagents used in each of these steps and their conditions of use vary depending on the particular situation and whether their use is required with any particular probes. Hybridization conditions are also described in U.S. Pat. No. 5,447,841. It will be appreciated that numerous variations of in situ hybridization protocols and conditions are known and may be used in conjunction with the present invention by practitioners following the guidance provided herein.

According to certain aspects, live cells are placed into growth media with a labeled probe for a period of time sufficient for the probe to internalize within the live cell and bind to a target nucleic acid sequence. Standard growth media and conditions for particular cells are well known to those of skill in the art. The time period for combining the labeled probe and the cell can be any desired time period. Exemplary time periods include 1 hour, 2, hours, 3 hours, 4 hours, 5 hours, 10 hours, 12 hours, 24 hours, 2 days, 7 days and longer if desired.

Cells may be washed according to methods known to those of skill in the art to remove unbound labeled probe and so as to reduce background signal. Suitable washing fluids are commercially available. Washing may also include centrifugation and resuspension one or more times in probe free media.

Probe concentration will vary depending on probe type, target size, and target complexity. For instance, LNAs have high affinity for their targets, and so would likely be applied at lower concentrations compared to other probes. Also, as repetitive targets are more easily detected, they would likely require lower concentrations of probe, even when targeted with smaller numbers of oligos, as compared to targets consisting of a unique sequence and, therefore, requiring complex libraries of oligos. Exemplary probe concentrations may be within the range of about 0.1 pmol to about 10 nm/ml. However, one of skill in the art will realize that useful concentrations may be outside of this range.

In order to improve probe hybridization efficiency, regions undergoing replication may be targeted as such regions are likely to be more easily dislodged from a complementary strand and/or associated factors such as proteins. Accordingly, methods include use of naturally occurring regions of replication which may occur in normally cycling cells. According to an additional aspect, methods include inducing replication shortly before adding the probe.

According to certain aspects, methods described herein may be used to visualize both repetitive genomic regions and single copy genomic regions. Different probes described herein may be selected.

For example, repetitive sequences may be targeted with as few as one and up to 10 or 20 ECHO probes, i.e. greater than 5, greater than 10, greater than 15, greater than 20, greater than 25, greater than 30, greater than 40, greater than 50, greater than 75, greater than 100 ECHO probes, etc., allowing the repetitive nature of the target to compensate for small number of probes. Single copy regions may be targeted with Oligopaints having a complementary probe sequence and a common binding sequence. A secondary oligo with an ECHO probe may be hybridized to the common binding sequence. This secondary labeling enables the use of complex oligo libraries while keeping the requirement for distinct ECHO probe species to a minimum.

Molecular beacons may also be used. Like ECHO probes, they fluoresce only when they bind to their target. Repetitive sequences may be targeted with as few as one and up to 10 or 20 molecular beacons, i.e. greater than 5, greater than 10, greater than 15, greater than 20, greater than 25, greater than 30, greater than 40, greater than 50, greater than 75, greater than 100 molecular beacons, allowing the repetitive nature of the target to compensate for small number of probes. Single copy regions may be targeted with Oligopaints having a complementary probe sequence and a common binding sequence. A secondary oligo with a molecular beacon may be hybridized to the common binding sequence. This secondary labeling enables the use of complex oligo libraries while keeping the requirement for distinct molecular beacon species to a minimum.

Toe-hold probes may also be used with reporter activation conditional upon binding to a target, as with a molecular beacon. Repetitive sequences may be targeted with as few as one and up to 10 or 20 toe-hold probes, i.e. greater than 5, greater than 10, greater than 15, greater than 20, greater than 25, greater than 30, greater than 40, greater than 50, greater than 75, greater than 100 toe-hold probes, allowing the repetitive nature of the target to compensate for small number of probes. Single copy regions may be targeted with Oligopaints having a complementary probe sequence and a common binding sequence. A secondary oligo with a toe-hold probe may be hybridized to the common binding sequence. This secondary labeling enables the use of complex oligo libraries while keeping the requirement for distinct toe-hold probe species to a minimum.

CRISPR technology may be used to target chromosomal DNA in vivo. Methods described herein provide for the synthesis of the Cas9 protein along with an RNA engineered to target the CRISPR system to designated chromosomal targets in mammalian (or other) cells and then additional strategies for making the targeted complex visualizable.

According to one aspect, methods are provided for synthesizing Cas9 in vivo include using an integrated construct, using a transiently transfected construct or by injection injection into the cell or a syncitia of nuclei (such as a Drosophila embryo) or via electroporation into cells and/or nuclei.

According to one aspect, methods are provided for synthesizing the RNA in vivo include using an integrated construct, using a transiently transfected construct or by injection injection into the cell or a syncitia of nuclei (such as a Drosophila embryo) or via electroporation into cells and/or nuclei.

According to one aspect, methods are provided for labeling the targeted Cas9/RNA complex by making a fusion protein that includes Cas9 and a reporter, such as GFP; by injection of RNA that has been attached to a reporter (e.g. fluorophore) into the cell or a syncitia of nuclei (such as a Drosophila embryo) or electroporation into cells and/or nuclei, or by indirect labeling of the RNA via hybridization with a labeled secondary oligonucleotide.

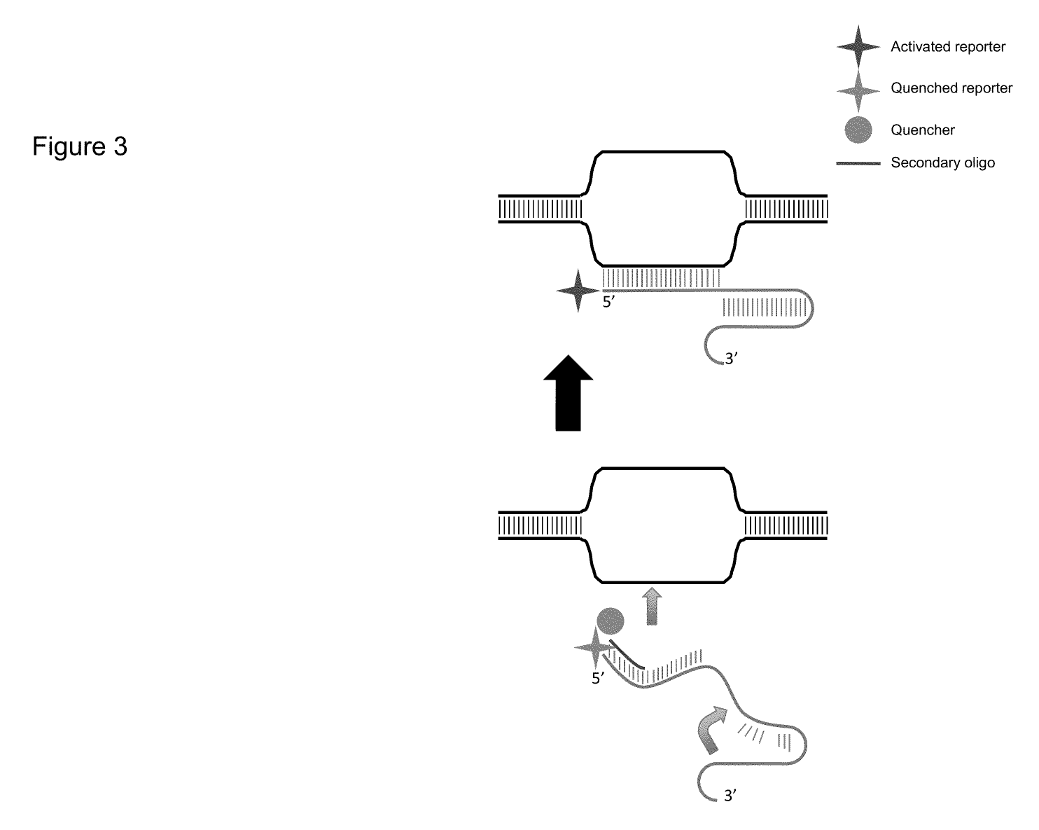

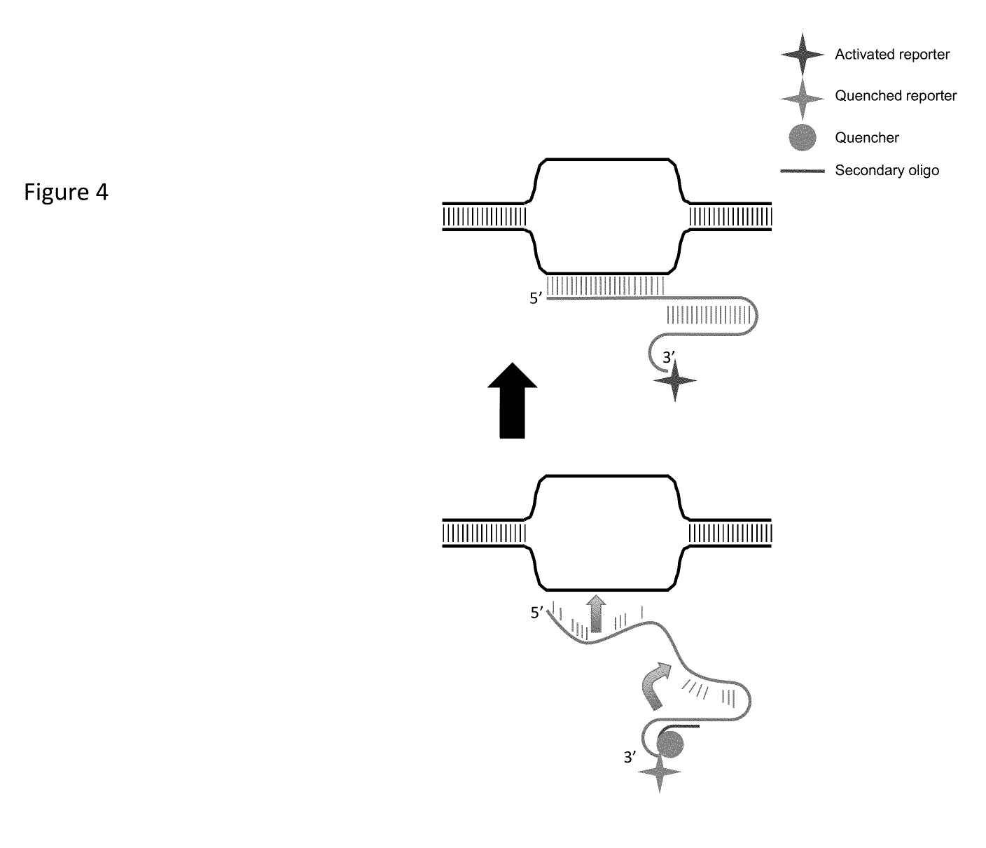

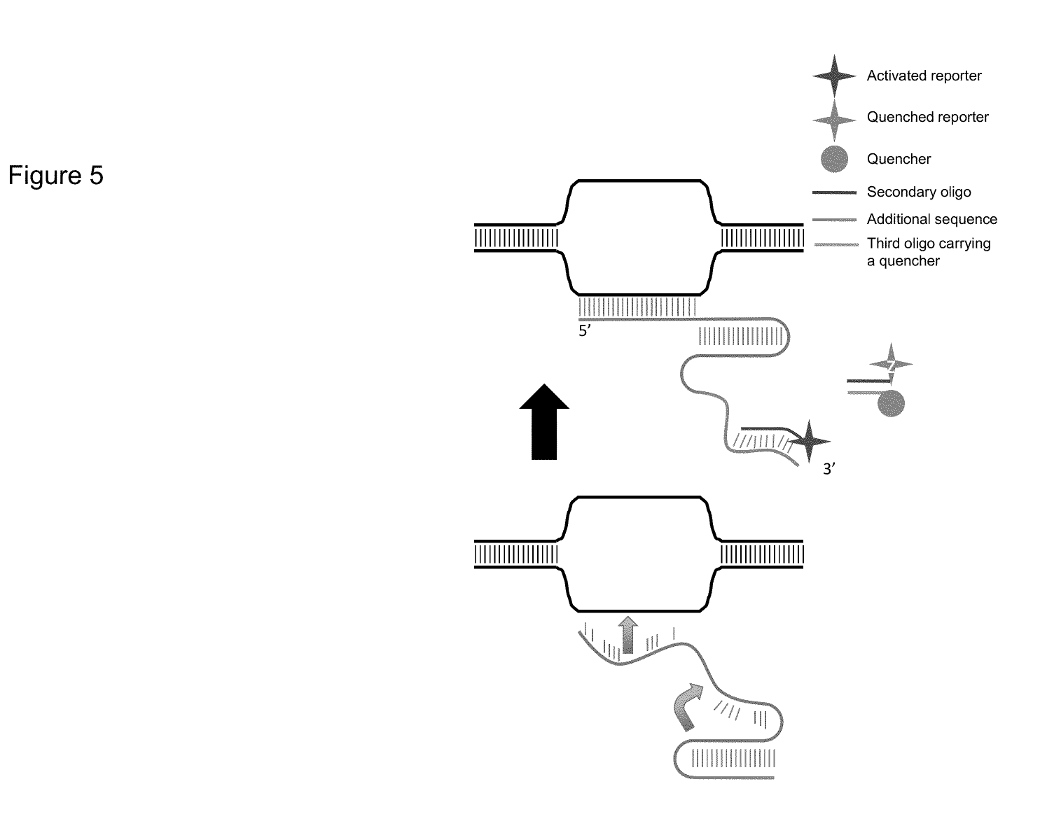

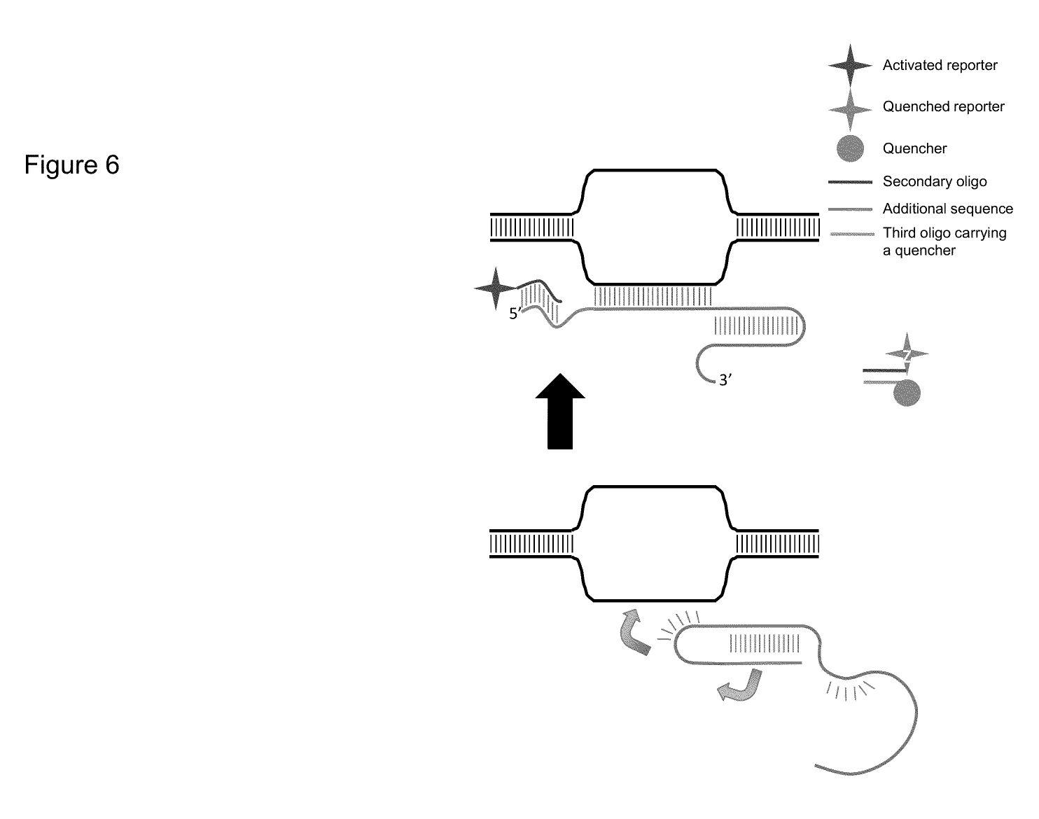

According to one aspect, methods are provided for making the reporter signal conditional by making a fusion protein that includes Cas9 and a reporter, with the reporter being conditional on the binding of the Cas9/RNA to the target. For example, if the binding of Cas9 and the RNA requires a specific conformation of these two components, then the reporter signal could be made dependent on that conformation via ‘split protein complementation’ (intramolecular complementation of multimers). This could involve the assembly of two or more proteinaceous parts, one or more proteinaceous parts with one or more RNA parts, or two or more RNA parts, with each part carrying one portion of the reporter. Further, the reporter can be made conditional by indirect labeling of the RNA via hybridization with a labeled secondary oligonucleotide, except that the activity/signal of the label (e.g., fluorophore) of the secondary oligonucleotide is conditional upon binding of the target. According to a particular aspect, a fluorophore (attached to the 5′ end of the RNA) is quenched via a secondary oligo until the RNA is bound to its target, whereby the secondary oligo with the quencher separates from the RNA and the fluorophore is activated. The reaction can be made to favor the targeted state by adjusting the length of the secondary oligo. Alternatively, a toehold could be used. According to this aspect, different secondary oligonucleotides could be used for each specific target. Such an exemplary embodiment is shown in FIG. 3. As shown in the alternate embodiment of FIG. 4, the fluor is attached to the 3′ end of the RNA. Again, it is quenched via a secondary oligo until the RNA has bound to its target. In this case, the sequence of the secondary oligo is not dependent on the target sequence. As shown in FIG. 5, the RNA has been extended at its 3′ end such that the added 3′ sequence is basepaired until the RNA has bound to its target. At this point, the added 3′ sequence is unpaired and available for binding by a secondary oligo bearing a fluorophore. Though not essential, this system can be enhanced through a third oligo that can bind the secondary oligo and quench its fluorophore when it is not bound to the RNA. This would reduce background fluorescence. A toehold could be used to enhance this system. As shown in FIG. 6, this system is similar to that shown and described with respect to FIG. 5, except it places the additional sequences at the 5′ end of the RNA.

According to an alternate embodiment, methods are provided for inserting targets for hybridization into a chromosome according to methods known to those of skill in the art. In this manner, a synthetic sequence having a strong hybridization affinity for a labeled probe may increase efficiency of probe hybridization. According to one aspect, the minimum number of bases to be inserted would be determined by the shortest sequence without endogenous homologs. Several systems are known for inserting sequences into a chromosome including CRISPR, TALENS and ZFN (zinc finger nucleases.) Once inserted, the probe may then be hybridized to the target. It is to be understood that the sequence inserted need not be limited to a single probe. The inserted sequence may contain any number of sequences complementary to a plurality of probes of the same or different type.

### Example I

### Culturing Live Cells in the Presence of Labeled Probes

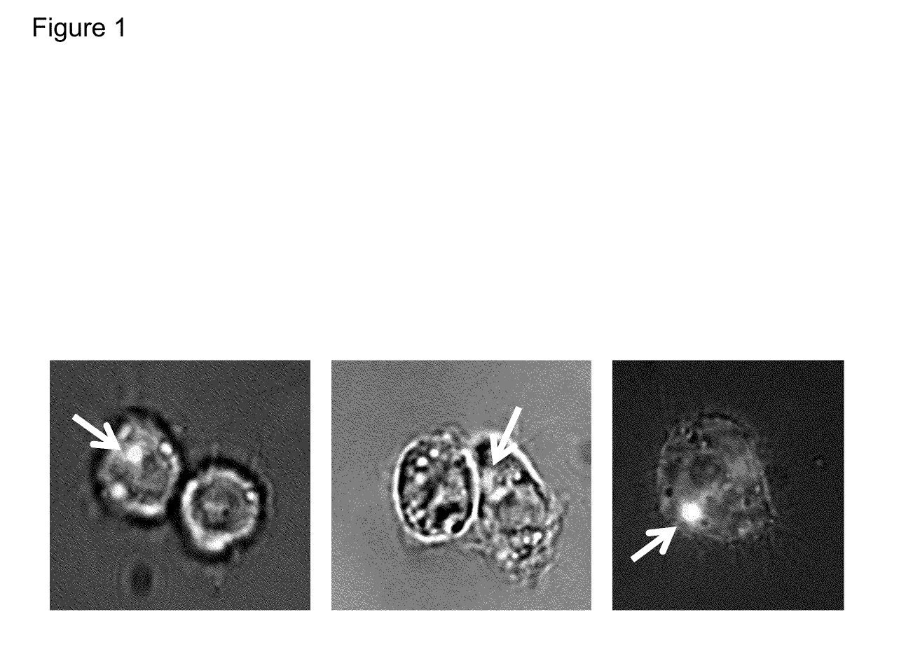

Drosophila Kc167 cells (1×10⁵ cells/ml) were placed into growth conditions with an LNA probe, dodeca labeled with Alexa488 for about 4 hours. The probe concentration was 30 pmol/μl and 3 μl per well was used for a total of 90 pmol of probe per well. The target nucleic acid was a centromeric dodeca repeat on chromosome 3. The cells were washed with 1×PBS and growth media was added for visualization with a microscope. Live cells were confirmed by visualization. As seen in FIG. 1, live cells were imaged showing the fluorescent probe within the live cells.

### Example II

### Culturing Live Cells in the Presence of a Labeled TALE

TALE technology enables the synthesis of proteins that bind theoretically any sequence in vivo. The proteins typically consist of arrays of highly similar domains, each of which is ˜33-35 aa in size and binds to a single base, the specificity of binding being determined by the aa present at positions 12 & 13 or thereabouts. TALEs may be fused with a reporter such as GFP so that the location of the target can be monitored within the live cell. TALES can be synthesized from templates that have been transiently transfected into a cell or integrated into the genome of the cell via standard technologies.

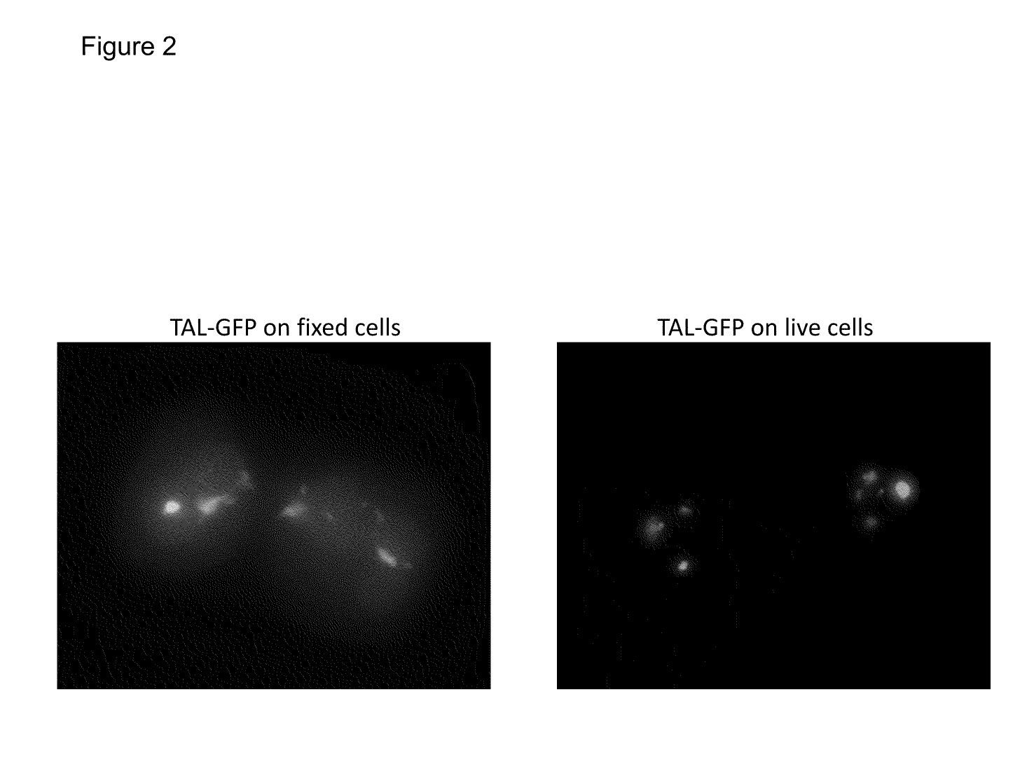

In this example, a TALE-GFP was designed to target the centromeric AACAC repeat on chromosome 2 of Drosophila, which has the sequence ‘AACAC’ repeated ˜500,000-1,000,000 times, i.e., AACACAACACAACACAACACAACACAACACAACACAACACAACACAACACAACAC etc. The layout of the protein is N-terminus/Nuclear Localization Sequence (NLS)/TALE domain/EGFP/C-terminus. A Drosophila Kozak consensus sequence upstream of the open reading frame was included. This insert was placed under the control of the pMT promoter. S2R+ were cultures using standard conditions at 25° C. S2R+ was transfected with the TALE-GFP using a commercial Effectine-based transfection kit following manufacturer's protocol. The cells were induced one day post-transfection using CuSO₄. Cells were allowed to continue to divide in the induction media following CuSO4 addition for multiple days. Transfectants were observed as early as 1 day post-induction (standard) as well as a few days afterwards. Imaging was done using a widefield epifluoresecent microscope on both live and fixed and mounted transfected cells. As seen in FIG. 2, live cells were imaged showing the TAL-GFP fluorescent probe within the live cells. TAL-GFP is also shown on fixed cells.

The contents of all references, patents and published patent applications cited throughout this application are hereby incorporated by reference in their entirety for all purposes.

### EQUIVALENTS

Other embodiments will be evident to those of skill in the art. It should be understood that the foregoing description is provided for clarity only and is merely exemplary. The spirit and scope of the present invention are not limited to the above example, but are encompassed by the claims. All publications, patents and patent applications cited above are incorporated by reference herein in their entirety for all purposes to the same extent as if each individual publication or patent application were specifically indicated to be so incorporated by reference.

What is claimed is:

1. A method of imaging a live cell by fluorescence in situ hybridization comprising
combining the live cell under growth conditions with a labeled probe having a sequence complementary to a genomic nucleic acid sequence, and
imaging the labeled probe within the live cell bound to genomic DNA.

2. The method of claim 1 wherein the probe is a labeled locked nucleic acid probe, a labeled oligonucleotide, an ECHO probe, a molecular beacon, a labeled toe-hold probe, a labeled TALE or a labeled Cas9/RNA complex.

3. The method of claim 1 further including the step of removing unhybridized probe from within the live cell.

4. The method of claim 1 further including the step of removing unhybridized probe from within the live cell by placing the cell in probe-free growth media and allowing the cell to double once or twice.

5. The method of claim 1 further including the step of removing unhybridized probe from within the live cell by centrifugation of the cell and resuspension in probe-free growth media.

6. The method of claim 1 wherein the genomic nucleic acid sequence is undergoing replication.

7. The method of claim 1 wherein the genomic nucleic acid sequence is undergoing naturally occurring replication.

8. The method of claim 1 wherein replication of the genomic nucleic acid sequence is induced before being contacted by the labeled probe.

10. A method of imaging a live cell by fluorescence in situ hybridization comprising
placing the live cell under growth conditions;
synthesizing a Cas9 within the cell,
synthesizing RNA within the cell to bind genomic DNA and to complex with the Cas9 forming a Cas9/RNA complex,
labeling the Cas 9/RNA complex, and
imaging the labeled Cas 9/RNA complex within the live cell bound to genomic DNA.

11. The method of claim 10 wherein the Cas9 is synthesized in vivo by using an integrated construct, a transiently transfected construct, by injection into the cell of a syncitia of nuclei or via electroporation into cells and/or nuclei.

12. The method of claim 10 wherein the RNA is synthesized in vivo by using an integrated construct, a transiently transfected construct, by injection into the cell of a syncitia of nuclei or via electroporation into cells and/or nuclei.

13. The method of claim 10 wherein the Cas9/RNA complex is labeled by making a fusion protein that includes Cas9 and a reporter, by injection of RNA that has been attached to a reporter into the cell or by a syncitia of nuclei including RNA that has been attached to a reporter, by electroporation into cells or nuclei or by indirect labeling of the RNA by hybridization with a labeled secondary oligonucleotide.

14. The method of claim 10 wherein the label is a conditional reporter.

15. The method of claim 10 wherein the label is a conditional reporter based on the binding of Cas9/RNA to the target nucleic acid.

16. The method of claim 10 wherein the label is quenched and is then activated upon the Cas9/RNA complex binding to the target nucleic acid.

17. A method of imaging a live cell by fluorescence in situ hybridization comprising
genetically altering the live cell to include one or more nucleic acid sequences complementary to a probe, combining the live cell under growth conditions with the probe having a sequence complementary to the one or more sequences added, and
imaging the probe within the live cell bound to one or more sequences added.

18. A method of making a live cell for in situ hybridization comprising
genetically altering the live cell to include one or more nucleic acid sequences complementary to a probe.


SEQUENCE LISTING (1 sequence)

SEQ ID NO: 1 - DNA, 55 bp, Artificial
  Target sequence
aacacaacacaacacaacacaacacaacacaacacaacacaacacaacacaacac